(19) United States
(12) Patent Application Publication
(10) Pub. No.: US 2021/0069113 A1
(43) Pub. Date: Mar. 11, 2021

(54) SOLID UNIT DOSAGE FORM FOR DOSE INDIVIDUALIZED DRUG DELIVERY

(71) Applicant: Modavar Pharmaceuticals LLC, Washington, DC (US)

(72) Inventor: Emad HASSAN, Germantown, MD (US)

(21) Appl. No.: 16/567,062

(22) Filed: Sep. 11, 2019

(51) Int. Cl. A61K 9/16; A61K 31/167; A61K 31/197; A61J 1/03; A61J 3/00

(52) U.S. Cl. CPC A61K 9/1694; A61K 9/1652; A61K 9/1635; A61K 9/1623; A61J 3/005; A61K 31/167; A61K 31/197; A61K 9/167; A61J 1/035; A61K 9/1617

(57) ABSTRACT

Embodiments disclosed herein relate to a package comprising a plurality of chambers, each of the plurality of chambers comprising a solid unit dosage comprising an active ingredient, wherein the package is configured to provide an individualized delivery of the active ingredient in different concentrations of the active ingredient to a human or an animal. The effective amount of active ingredient is adjusted in the solid unit dosage according to the size or weight of solid unit dosage. The manufacture of the solid unit dosage comprises mixing of the active substance with pharmaceutically acceptable additives, followed by hot or cold extrusion and spheronization. The solid unit dosages can be manufactured without the use of a tablet press, lyophilization or molding. Solid unit dosages with various drug loads or many drug strengths simultaneously are produced in one batch without further processing.

### TECHNICAL FIELD

The presently disclosed embodiments relate to compositions for solid unit pharmaceutical dosage forms, methods of making and their utilities. An embodiment finds its utility in individualizing or personalizing the dose for each patient without the need to additional prescriptions. An embodiment relates to compositions, processes and uses of new solid unit dosage systems (SUDs) that can be manufactured without the use of a tablet press, lyophilization or molding. An embodiment further provides a process that generates solid unit dosages with various drug loads or many drug strengths simultaneously in one batch without further processing.

### BACKGROUND

Solid unit dosages are most abundant forms used for drug administration. A solid unit dose is the amount of a medication administered to a patient in a single dose, but in a solid form. They are all made of multiparticulate systems as an intermediate bulk product. Multiparticulate systems include powders, SUDs, micro SUDs, microspheres, nanoparticles, microcapsules, pellets, micro-pellets, granules. The common methods of preparing such systems are wet/dry granulation and hot/cold extrusion. In all cases, those intermediate products must be further processed through additional manufacturing steps such as compression into a tablet or filling in a capsule shell or a sachet to make a finished unit dose, ready for direct administration.

“The preparation of microparticles is mainly used so as to delay the dissolution of active principles and, because of this, finds numerous applications in the field of controlled-release medicaments and in the field of masking the taste of medicaments intended for oral administration. However, it has always been difficult to develop a formulation containing microparticles in the form of unit doses and in particular a formulation suitable for oral administration.” [Source: U.S. Pat. No. 5,417,985].

On the other hand, mini-tabs are merely small tablets (1 mm-4 mm) that pass through all steps of tablet manufacturing and then filled basically as a group of particles to provide a dose. In other words, none of the above multiparticulate systems provides a directly usable unit dosage form that a patient can easily hold and self-administer. Furthermore, it is not yet known how to use the all above mentioned dosage forms directly as a one solid unit dosage form or how to manufacture many drug strengths simultaneously in one batch without further processing. In case of manufacturing tablets, a significant compressional force must be applied to granules, powders pellets to form the unit dose. Some drugs would lose part of its efficiency if made into tablets, because of the damaging nature of compressional forces. Furthermore, compressing coated multiparticulates could cause damage to the particle's coat and thus reduce the efficiency of functional coatings. While filling multiparticulate systems into capsules avoids the exposure of particles to excessive forces, it adds one more step (capsule filling) to the manufacturing process which automatically increases the cost of goods.

Many life-saving drugs have narrow therapeutic indexes or windows where patients may receive ineffective under-dose or a life-threatening overdose. This huge medical hurdle is further complicated by the fact that some of those drugs have a variable absorption in humans due to inherited, or acquired, physiological or pathological changes. “Conventional drug delivery systems, such as immediate release drug delivery systems, have only limited use for: (1) active agents having an absorption window in the gastrointestinal tract; (2) active agents which have a locus of treatment in or proximal to the gastrointestinal tract; and (3) active agents which degrade in the colon.” [Source: U.S. Pat. No. 8,007,827 B2]. With current dosages, personalized or individualized dosing is economically and clinically difficult. In theory, an almost continuous range of drug amounts that are all bioequivalent requires the development of a perfectly accurate dosage form that can be precisely measured and administered to a patient without the need of a health-care giver assistance (i.e. hospitalization or nurse visits to patients). As known by those experienced in the field, liquid dosage forms can be used to provide variable drug amounts. However, errors of administration happen because of the design or the use of inaccurate measuring tools. Moreover, certain medicaments are less stable in liquid dosage forms than in solid dosage forms. Also, the overall cost of manufacturing and transportation of liquid dosage forms is significantly higher than solid dosage forms. Breakage, leaking and inherent higher weight of liquid dosage forms leaves solid dosage forms such as tablets and capsules to be the most popular in the health care industry.

Alternatively, another avenue to personalizing drug dosing can be by manufacturing numerous strengths of unit solid dosage forms such tablets or capsules. An incremental increase in doses to provide more flexibility to the health care giver to tailor a therapeutic regimen for individual patients sounds possible. However, the approach of making numerous doses of one solid drug will increase the number of batches needed to cover a wide dose range and therefore extended periods of time and increased quality control and quality assurance costs which, in return, will result in an overall increase in price of medications.

Hormone therapy medications like contraceptives and their placebos are available in one packaging, but not in the dose individualized form. There is a need to package medicines with varied individualized drug dosages in the same package. Unit dose packaging can also help patients maintain a proper use of medication, and therefore have a proper healing process.

Usually dose personalization is a time-consuming and is sometimes impractical when tablets are used to individualize a patient's treatment. Apart from the cost attached to manufacturing many tablet strengths, the dose cannot be accurately administered, once a patient starts to use a half or a fraction of a tablet to get a prescribed drug amount.

Another theoretical alternative to personalize drug doses is to use the drug in a form of liquid. However, very few drugs are naturally liquid in nature. Making a solution or a suspension of a solid drug appears as a logic choice and can be generally with better bioavailability relative to solid dosage forms. However, stability of drugs is usually less when drugs are in the liquid form than in the solid form. Liquid dosage forms also are more expensive to make and less convenient while travelling.

Patent No. CA2520660C describes a soft gelatin dosage form. It is built on a core liquid that would take the shape of an outer layered congealing gelatin. This patent is therefore limited to using of gelatin and in fact does need a complicated encapsulation machine. The fact that a liquid must fill the shell to make it take the shape of the shell would require the core to be fluid in nature with high amount of liquid, producing a dosage form that may leak if the shell is not perfectly sealing the drug mixture.

Patent No. CN101951891A provides fine particles with high drug load medicament, preferably comprising one or more oral compositions of active pharmaceutical ingredient including acetaminophen, using extrusion techniques. The reference teaches a standard hot melt extrusion but does not teach particle size control and requires the extruded particles to be filled to capsules or tablets.

The inability of satisfying the un-met medical need of personalizing or tailoring drug dosing using a solid unit dosage continues to be a threat to public health. In a world where so many people are taking prescription medications daily, it is beneficial for patients to have their medications pre-packaged in unit doses. To overcome the drawbacks of existing dosage forms in providing the un-met medical need of dose individualization, an embodiment formulates solid unit dosage systems with different strengths that will help doctors in the titration of patient dose. It would be much easier for the patient to comply with doctors' prescribing instructions and obtain a more effective, more economic and a speedy treatment.

### SUMMARY

An embodiment relates to a package comprising a plurality of chambers, each of the plurality of chambers comprising a solid unit dosage comprising an active ingredient, wherein the package is configured to provide an individualized delivery of the active ingredient in a plurality of different concentrations of the active ingredient to a human or an animal, wherein a concentration of active ingredient in the solid unit dosage in each of the plurality of chambers is different, wherein the plurality of different concentrations of the active ingredient comprise at least two different concentrations that are greater than zero weight percent of the active ingredient.

In an embodiment, wherein an effective amount of the active ingredient is present in the solid unit dosage according to a size or a weight of the solid unit dosage.

In an embodiment, the solid unit dosage is neither a capsule nor a tablet.

An embodiment relates to a solid unit dosage comprising an active ingredient dispersed in a suitable medium, wherein an effective amount of the active ingredient is present in the solid unit dosage according to a size or a weight of the solid unit dosage such that the solid unit dosage is configured to provide an individualized delivery of the active ingredient to a human or an animal.

Another embodiment refers to the solid unit dosage further comprises a safe and effective amount of at least one substance selected from the group consisting of a filler, a carrier, a binder, a disintegrant, a surface active agent, a lubricant and combinations thereof.

The solid unit dosage further comprises a coating material.

The active ingredient comprises a material comprising a therapeutic agent, a diagnostic agent, a food supplement or combinations thereof, and the material is present in an amount of 0.01 to 80 percent by weight of the solid unit dosage.

The suitable medium comprises a volatile liquid or a non-volatile liquid and is present in an amount of 1 to 75 percent by weight of the solid unit dosage.

The volatile liquid is selected from the group consisting of water, methanol, ethanol, 1-propanol, isopropyl alcohol and combinations thereof.

The non-volatile liquid comprises polyethylene glycol.

The filler is selected from the group consisting of mannitol, lactose, a polymeric saccharide, starch, polyvinylpyrrolidone and combinations thereof, wherein the filler is present in an amount of 0.01 to 40 percent by weight of the solid unit dosage.

The carrier is selected from the group consisting of silicon dioxide, zinc oxide, magnesium oxide, a porous natural polysaccharide, a synthetic polysaccharide and combinations thereof, wherein the carrier is present in an amount of 0.01 to 50 percent by weight of the solid unit dosage.

The binder is selected from the group consisting of hydroxypropyl methylcellulose, polyvinyl pyrrolidone, methyl cellulose, gelatin, starch, sucrose, lactose and combinations thereof, wherein the binder is present in an amount of 0.01 to 30 percent by weight of the solid unit dosage.

The disintegrant is selected from the group consisting of cross-caramellose, cross-povidone, a modified starch, sodium starch glycolate and combinations thereof, wherein the disintegrant is present in an amount of 0.01 to 15 percent by weight of the solid unit dosage.

The surface active agent comprises polaxmer, wherein the surface active agent is present in an amount of 0.01 to 50 percent by weight of the solid unit dosage.

In another embodiment, the solid unit dosage has the following properties: a sphericity factor of about 0.9 to about 1.0; a hardness more than 10 kilograms; a diameter in a range from 1.5 to 6 mm; a weight in a range from 15 to 300 mg.

In an embodiment, wherein at least one of the plurality of chambers comprises a placebo having zero weight percent of the active ingredient.

In an embodiment, the plurality of chambers comprises at least three chambers.

An embodiment relates to a method comprising mixing an active ingredient in a suitable medium, and forming a solid unit dosage by extrusion and spheronization, wherein the solid unit dosage comprises the active ingredient dispersed in the suitable medium, wherein an effective amount of the active ingredient is present in the solid unit dosage according to a size or a weight of the solid unit dosage such that the solid unit dosage is configured to provide an individualized delivery of the active ingredient to a human or an animal.

The method further comprises drying using vacuum, heat, air, or any pharmaceutically acceptable inert gas.

The method further comprises coating the solid unit dosage.

The method comprises a continuous process or a batch process that simultaneously produces at least two strengths of the active ingredient.

An embodiment relates to a solid unit dosage form for dose individualized delivery of an active ingredient comprising the active ingredient dispersed in a suitable medium, further comprising safe and effective amounts of at least one substance selected from the group consisting of filler, carrier, binder, disintegrant and surface active agent, wherein the effective amount of active ingredient is adjusted in the solid unit dosage form according to the size or weight of solid unit dosage.

Another embodiment relates to a solid unit dosage form for dose individualized delivery of an active ingredient comprising 0.01 to 80 percent by weight units of the active ingredient, 0.0 to 40 percent by weight of a filler, 1 to 75 percent by weight of volatile or non-volatile liquids, 0.1 to 50 percent by weight of a carrier, 0.01 to 30 percent by weight of a binder; 0.01 to 15 percent by weight a disintegrant, 0.01 to 50 percent by weight of a surface active agent, an optional lubricant; and an optional coating, wherein the diameter of solid unit dosage ranges from 1.5 to 6 mm, with a sphericity factor of 0.9 to 1.0 and a hardness of more than 10 kilograms, and wherein the effective amount of active ingredient is adjusted in the solid unit dosage form according to the size or weight of solid unit dosage.

Yet another embodiment relates to a method comprising mixing an active ingredient in a suitable medium, and forming a solid unit dosage by extrusion and spheronization, wherein the solid unit dosage comprises the active ingredient dispersed in the suitable medium, wherein an effective amount of the active ingredient is present in the solid unit dosage according to a size or a weight of the solid unit dosage such that the solid unit dosage is configured to provide an individualized delivery of the active ingredient to a human or an animal. The method includes the steps of mixing of the active substance with pharmaceutically acceptable additives, followed by hot or cold extrusion and spheronization, optionally followed by a drying step. The solid unit dosage forms may further be coated to provide additional function such as immediate, enteric, delayed or pulsatile or controlled release of the drug. Additionally, aesthetic properties such as color, smoothness shining, or general elegance of the dosage can be altered with optional coating.

Another embodiment relates to the process wherein the process can be implemented in a continuous or in a batch manner and simultaneously produces at least two strengths of the active ingredients.

Yet another embodiment relates to a delivery system comprising a solid unit dosage comprising an active ingredient, wherein the delivery system is configured to provide an individualized delivery of the active ingredient in a plurality of different concentrations of the active ingredient to a human or an animal, wherein a concentration of the active ingredient in the solid unit dosage within the delivery system is different, wherein the plurality of different concentrations of the active ingredient comprise at least two different concentrations that are greater than zero weight percent of the active ingredient.

### DETAILED DESCRIPTION OF SOME EMBODIMENTS

### Definitions and General Techniques

All publications, patents, and patent applications cited in this Specification are hereby incorporated by reference in their entirety.

The articles “a” and “an” are used herein to refer to one or to more than one (i.e., to at least one) of the grammatical object of the article. The term “solid unit dosage” means the amount of a medication administered to a patient in a single dose, but in a solid form which may contain a polymer or more than one polymer. The term “solid unit dosage” is sometimes abbreviated as SUD and has been occasionally used in this Specification. The term “sphericity” is defined as the ratio of the surface area of a sphere having the same volume as the particle to the surface area of the particle. The term “hardness” is a measure of the resistance to localized plastic deformation induced by either mechanical indentation or abrasion. The term “dose individualization” is defined as the adaptation of the dosage regimen in function of the clinical characteristics of the individual, aiming to achieve the best possible therapeutic efficiency at the lowest risk of unwanted effects. Hence “dose individualized delivery” would mean delivery of the active ingredient as per dose individualization. The term “drug loading” means the amount of drug or active ingredient, present in a single SUD.

Any ranges cited herein are inclusive. The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

An embodiment describes a solid unit dosage (SUD) for dose individualized delivery of an active ingredient, comprising the active ingredient dispersed in a suitable medium, further comprising safe and effective amounts of at least one substance selected from the group consisting of filler, carrier, binder, disintegrant and surface active agent, wherein the effective amount of active ingredient is adjusted in the solid unit dosage form according to the size and weight of solid unit dosage.

The solid unit dosage may further contain an optional lubricant; and probably an optional coating. An embodiment produces SUDs in form of beads or megabeads. The term “SUDs” may be interchangeably used with “beads” or “megabeads”.

The drug loading of a single SUD may range from 0.01% to 80% of the active ingredient. The active ingredient can be a therapeutic agent, a diagnostic agent, a food supplement or combinations thereof.

A filler is defined as an inactive substance used to make the active ingredient easier to measure or homogenously distribute in a SUD. Fillers are often used in tablets or capsules because the amount of active drug is too small to be handled conveniently. The filler in an embodiment may range from 0.01 to 40% of weight of the SUD. Some examples of fillers that may be used are mannitol, lactose, polymeric saccharides, starch and polyvinylpyrrolidone (PVP).

A “volatile liquid” is defined as a liquid that boils at low temperature (up to 100° C.), or changes from liquid to gas at low temperatures. A “non-volatile liquid” is defined as liquid that has high boiling temperatures (more than 100° C.). Volatile and/or non-volatile liquid in an embodiment may range from 1 to 75% of weight of the SUD, when wet, e.g. water, methanol, ethanol, 1-propanol, isopropyl alcohol, or propylene glycol, liquid sugars or solutions of sugars.

A binder/binder solution may be selected from a group consisting of cross-linked polymers such as cross-linked polyvinylpyrrolidone (PVP), modified starch and hydroxypropyl methyl cellulose (HPMC) or derivatives thereof, and concentration of the binder may vary from 0.01 to 30% of weight of the SUD.

A disintegrant is an agent used in pharmaceutical preparation of tablets, which causes them to disintegrate and release their medicinal substances on contact with moisture. Disintegrants used in an embodiment may vary from 0.01 to 15% of weight of the SUD. Cross linked polymers such as cross-caramellose and/or cross-povidone, modified starch, sodium starch glycolate can be used as disintegrants.

The SUDS of an embodiment may also carry water-insoluble polysaccharide such as microcrystalline cellulose in the range of 5 to 95% by weight of the SUD.

A drug carrier/carrier is any substrate used in the process of drug delivery which serves to improve the selectivity, effectiveness, and/or safety of drug administration. Drug carriers are primarily used to control the release of a drug into systemic circulation. The carrier such as silicon dioxide polymers and are in the range of 0.1 to 50% by weight of the SUD. Other examples of carriers are zinc oxide, magnesium oxides, or porous natural or synthetic polymers such as natural or synthetic polysaccharides.

Surface active agents, in solid or liquid form, such as polaxmers may be added in a concentration of 0.01 to 50% by weight of the SUD.

Optional lubricants such as stearic acid, magnesium stearate, talc, or polyethylene glycol of solid nature may be added in the range of from 01% to 5%. The SUD may be optionally coated with a film coated for immediate or controlled release. Without limiting the invention to one mechanism of drug release, SUDS can release the drug in immediate, controlled, or delayed release or pattern. The coat can provide additional functions such as immediate, enteric, delayed or pulsatile or controlled release of the drug. Additionally, aesthetic properties such as color, smoothness shining, or general elegance of the dosage can be altered with optional coating. Anti-oxidants or stabilizers may also be added to prepare the SUD composition.

The solid unit dose (SUD) can range from 1.5-6 mm in diameter, is spherical or almost spherical made with a sphericity factor of 0.90 to 1.0. The hardness of a SUD is more than 10 kilograms. The effective amount of active ingredient is adjusted in the solid unit dosage form according to the size and weight of solid unit dosage. The SUD is small enough to be easily swallowed and large enough to be easily handled by patients.

An embodiment further claims the use of such dosage form to titrate patients with drugs that have narrow therapeutic windows or of high variability in absorption such as digoxin, phenytoin, theophylline, immunosuppressants and other small or large molecules which have a narrow therapeutic index or window.

In one embodiment, SUDs are spherical or almost spherical particles of about 1.5 mm in diameter to about 6 mm in diameter. Preferably SUDs will have a diameter of 2.0 to 5.5 mm, more preferably between 2.8 mm and less than 5 mm.

In yet another embodiment, SUDs have a weight from approximately 15 mg to approximately 300 mg, preferably, from 20 mg to 200 mg, more preferably from 25 mg to 150 mg.

An embodiment also teaches how to consistently have accurate dosing based on volume of the homogenous mass of the whole particle population. The described SUDs can contain volatile and non-volatile liquids up to 60% of its weight when wet and up to 30% of its weight of liquid when dry yet had a hardness of not less than 10 kilograms as measured by the method explained in the examples.

In another embodiment the drug is present as a solution in one or more liquids. A drug solution can be a true solution, a micellar solution or with the aid of liposomes and/or surface-active agents or surfactants.

In another embodiment, the active ingredient may be in a solid state suspended in a liquid vehicle.

In one embodiment, the structure of SUDs will disintegrate within 3 minutes.

In another embodiment, SUDs can be formulated using a carrier system consisting of a filler, an adsorbent, a binder, a disintegrant, a non-volatile liquid and an aqueous of non-aqueous fluid, and optionally a volatile liquid, a color and/or lubricant.

In another embodiment, fluid used in the manufacturing of the SUDs can be volatile or non-volatile.

In another embodiment SUDs are soft or hard in texture. Soft SUDs possess elastic or plastic mechanical properties by having one or more non-volatile solvent. Examples for non-volatile solvents are dimethyl sulfoxide, liquid dihydroxyl or poly-hydroxyl alcohols, such as glycerol, polyethylene glycol, propylene glycol, or a mixture thereof.

In an additional embodiment, SUDs can use water, or other suitable liquid vehicles containing no water at a concentration of 1-99% of the SUD weight, preferably 2% to 50% and more preferably 5%-50%.

In yet an additional embodiment, the fluid is made of volatile and/or non-volatile liquid mixture of aqueous and/or non-aqueous mixture at a concentration of 1-75% w/w, preferably 5-60% and more preferably 10-50% of the SUD's weight.

Not only does the current invention find its utility to individualize dosing of drugs, but it is also extremely useful for patients who are having difficulties in swallowing conventional solid unit doses (SUDs). Geriatric, pediatric or other patients who cannot swallow trivial solid unit dosage forms such as tablets or capsules.

An embodiment further describes the process of making the solid unit dosages. It does not use tableting compression where the active ingredient may lose some or all of its activity or potency. This process includes the mixing of all the required ingredients in a way described in the Examples section, followed by hot or cold extrusion, and then spheronization to create spherical SUDs. Spheronization is a method to create spherical or almost spherical particles. An embodiment does not use tableting compression, lyophilization or molding, where some molecules can lose some or all their activities. The process of making an embodiment also uses extrusion methods (hot or cold) according to the drug stability. After extrusion, the population of SUDs are then classified according to weight or volume to provide multiple strengths of the drug from the same batch.

In one embodiment, the invented SUDs may not need drying and can be directly administered to animals or pediatric, geriatric or middle-aged humans.

In another embodiment, a drying step can be applied. Drying can be performed using a fluid bed or oven drying.

In one embodiment, SUDs can be directly administered to patient without coating or drying.

SUD coating can be achieved using a coating pan, or a fluid bed. Coating can be applied to provide additional functional or aesthetic properties or characteristics. The invented SUDs can be used for small or large active molecules that are physically or chemically sensitive to drastic conditions such as tableting compression, heat, and/or freezing.

According to an embodiment, SUDs can be produced continuously or in a batch manner and simultaneously produces at least two strengths of the active ingredients.

One embodiment to make extrusion the resultant material can be spheronized using a rotating plate or spheronizer.

In another embodiment drying of the SUDs is performed using vacuum, heat, or air or any pharmaceutically acceptable inert gas such as nitrogen or helium. Drying equipment can be ovens or fluid bed driers.

SUDs can also be easily disintegrating in the mouth or in suitable palatable liquids or semisolids to administer to an animal or a human of any age.

For immediate release, disintegration or erosion of the SUD occurs in less than 15 minutes, preferably less than 10 minutes and more preferably less than 5 minutes.

Transforming SUDs into a pulsatile delayed or controlled release dosage form is well known to those who are trained in the field. SUDs can be delayed release when coated by an acid insoluble material or substantially acid insoluble materials which are collectively called enteric materials or polymers such as waxes, fatty acids, cellulose acetate phthalate, cellulose acetate succinate, hydroxypropyl methyl cellulose (HPMC) acetate, HPMC phthalate, HPMC acetate phthalate, sodium alginate cross linked gelatin, cross linked the phthalate or succinate esters of cellulosic polymers such as polyacrylic acid polymers.

A therapeutic dose can exist in 1, 2, 3 or 4 SUDs of similar or different drug strengths during dose titration. After identifying the sustaining or constant dose, patients can take one or more SUDs of similar or different strengths.

The preferred dosage for the SUDs is 1-4 SUDs. A more preferred number to be administer as a dose is 1-3 SUDs and yet more preferably 1-2 SUD. The most preferred dosage is to use one SUD.

Another objective is an effective drug delivery system for delivering single unit dosages. Drug delivery refers to approaches, formulations, technologies, and systems for transporting a pharmaceutical compound in the body as needed to safely achieve its desired therapeutic effect.

Yet another objective is a drug delivery system configured to provide an individualized delivery of the active ingredient in a plurality of different concentrations of the active ingredient to a human or an animal, wherein a concentration of the active ingredient in the solid unit dosage within the delivery system is different, wherein the plurality of different concentrations of the active ingredient comprise at least two different concentrations that are greater than zero weight percent of the active ingredient.

The drug delivery system involves packaging the unit dosages with various concentrations in multiple chambers or in a plurality of chambers but in the same package. A package can be defined as the final product that contains the drug or the solid unit dosage in various pockets or chambers, and as handed out to end user. The chambers are the pockets where the solid unit dosage or the drug is stored. The package can be made of any pharmaceutically acceptable material dependent on the active ingredient and the final method of drug delivery.

Yet another objective is a package comprising a plurality of chambers, each of the plurality of chambers comprising a solid unit dosage comprising an active ingredient, wherein the package is configured to provide an individualized delivery of the active ingredient in a plurality of different concentrations of the active ingredient to a human or an animal, wherein a concentration of the active ingredient in the solid unit dosage in each of the plurality of chambers is different, wherein the plurality of different concentrations of the active ingredient comprise at least two different concentrations that are greater than zero weight percent of the active ingredient.

### Advantages

Advantages of the disclosed embodiments include providing a solid unit dosage for individualized dose delivery of the active ingredient.

An embodiment is a new path of drug therapy that introduces desired therapeutic effect, with the highest chance of efficacy and minimum toxic effects, by producing different dose loaded SUD (bead)s. SUDs of an embodiment allow the opportunity for doctors/pharmacists to choose the correct titration of patient dose. It would be much easier for the patient to comply with doctors' prescribing instructions and obtain a more effective, more economic and a speedy treatment.

The process of preparing the SUD provides more than one strength in a single batch of SUDs, thus reducing the cost of production.

Another embodiment is a drug delivery system configured to provide an individualized delivery of the active ingredient in a plurality of different concentrations of the active ingredient. The system involves packaging the unit dosages with various concentrations in multiple chambers but in the same package.

### EXAMPLES

Those who trained in the art can use methods obvious to incorporate drugs in the following examples similar to the following

The examples below describe the preparation and evaluation methods for SUDs. The drug can be either in solution i.e. dissolved in the liquid components of the SUD where the solvent used can act as a model of the drug containing liquid. It can also be a water soluble solid where the SUD's ingredient mannitol can be a model material. The drug can also be water insoluble drug where the Avicel or Aerosil can represent that category of drugs.

### Example 1

Composition Using Non-Volatile Solvent Ethanol

A 600 g batch of SUDs was prepared using microcrystalline cellulose (AVICEL PH 101) (42% w/w), mannitol (20% w/w), cross-linked polyvinylpyrrolidone (crospovidone) (5% w/w), Polyvinylpyrrolidone (PVP, Molecular weight 30 kilodaltons) (3% w/w), Propylene Glycol (PG) (30% w/w), and Ethanol (30% w/w).

Method of Preparation

18 g of PVP K30 (PVP; k number refers to the mean molecular weight of PVP) was dissolved in 180 grams of ethanol (200 proof) and 180 grams of propylene glycol using an HSM-100 Ross High Shear Mixer (Charles Ross and Son Company, 710 Old Willets Path, Hauppauge, N.Y. 11788 U.S.A.) to form a binding solution. After 60 seconds of dry mixing AVICEL PH 101, mannitol and crospovidone in a 6 Quart Professional 600 Series Planetary Mixer (Kitchen Aid, 553 Benson Rd, Benton Harbor, Mich. 49022 U.S.A.), the binding solution was poured into the mixer at a constant rate over 90 seconds, while mixing at speed 2. Mixing was continued at speed 2 for 30 seconds. The wet mass was immediately extruded at room temperature through a 4 mm die using an MG-55 Multi Granulator Extruder (Fuji Paudal, 7-1-45 Nakaishikiri-Cho Higashi Osaka, 579-8014 Japan). The output of the extrusion step was spheronized using a QJ-230T Spheronizer (Fuji Paudal, 7-1-45 Nakaishikiri-Cho Higashi Osaka, 579-8014 Japan), fitted with a 5 mm friction plate for 4 minutes. Resultant SUD (bead)s were dried in a fume hood at room temperature for 48 hours. Dried SUDs were then classified using a set of sieves and an RX-30 RO-TAP Sieve Shaker (W. S. Tyler, 8570 Tyler Blvd, Mentor, Ohio 44060 U.S.A.). Sieves were 12″ in diameter ranging from 1.7 mm to 5.6 mm (Hogentogler, 9515 Gerwig Ln #109, Columbia, Md. 21046 U.S.A.).

### Example 2: Evaluation of SUDs

Dried SUDs from Example 1 were evaluated for the following:

### A. Yield:

The yield of each size class is shown in Table 1.

(The preferred size of SUDs is 2.8 mm-4.75 mm)

TABLE 1
The % yield of the SUDs of various sizes

| | |
|---|---|
| Diameter of SUDs | % Yield |

| | | |
|---|---|---|
| >5.60 | mm | 0.2% |
| 4.75-5.60 | mm | 2.3% |
| 4.00-4.75 | mm | 20.0% |
| 3.35-4.00 | mm | 30.5% |
| 2.80-3.35 | mm | 20.4% |
| 2.36-2.80 | mm | 10.1% |
| 2.00-2.36 | mm | 6.0% |
| 1.70-2.00 | mm | 3.7% |
| <1.70 | mm | 6.7% |

### B. Hardness of SUDs:

Hardness testing of SUDs from Example 1 was performed using a TA.XT Plus Texture Analyzer (Stable Microsystems, 6 Patton Drive, South Hamilton, Mass. 01982 U.S.A.) equipped with a flat cylinder probe. The hardness of SUDs from the preferred size range (2.8-3.35 mm, 3.35-4 mm and 4-4.75 mm size range) is recorded as shown in Table 2.

TABLE 2
Hardness of SUDs with preferred size classes from example 1

| Diameter of SUDs | Hardness |
|---|---|
| 4.00-4.75 mm | 28.98 ± 1.60 kg |
| 3.35-4.00 mm | 23.60 ± 2.36 kg |
| 2.80-3.35 mm | 18.93 ± 1.32 kg |

Example 3-10 below present the effect of different volatile liquids on the physical properties of the SUDs.

### Example 3

Composition and Method of Manufacturing

In Example 3, the composition of the SUDs uses methanol instead of ethanol (as in Example 1). All other steps and procedures remain the same as in in Example 1. Table 3 below shows the composition of the various ingredients.

TABLE 3
Formulation composition for batches
manufactured with Methanol: PG

| Ingredient | Weight Units |
|---|---|
| AVICEL PH 101 | 42 |
| Mannitol | 20 |
| Crospovidone | 5 |
| PVP K30 | 3 |
| Propylene Glycol (PG) | 30 |
| Methanol | 30 |
| Total | 100* |

*Please note that the total is 100 because the 30 weight units contributed by the volatile solvent (methanol) is evaporated off while drying. Therefore, the effective final SUD does not contain any volatile solvent.

### Example 4

Evaluation of SUDs of Example 3

Dried SUDs from Example 3 were evaluated for the following:

### A. Yield

Classification and yield for SUDs from Example 3 were performed as stated in Example 2. The particle size results for SUDs from Example 3 is shown in Table 4.

TABLE 4
The % yield of the SUDs of various sizes

| Sieve Size Classification | % of SUD |
|---|---|
| (mm) | (bead) weight |
| >5.60 | 0.0 |
| 4.75-5.6 | 3.3 |
| 4.00-4.75 | 47.8 |
| 3.35-4.00 | 28.4 |
| 2.80-3.35 | 11.4 |
| 2.36-2.80 | 4.0 |
| 2.00-2.36 | 1.7 |
| 1.70-2.00 | 0.8 |
| 1.70> | 2.5 |

The combined % yield for the preferred size range (>2.8 mm to <5.6 mm) is 90.9%.

### B. Hardness of SUDs

Hardness of SUDs from Example 3 were tested as stated in Example 2. The hardness of SUD (bead)s from the 3.35-4.00 mm and 4.00-4.75 mm size range can be found in Table 5.

TABLE 5
SUDs hardness of preferred size ranges of
Example 3; RSD: Relative standard deviation

| | SUDs stayed | SUDs stayed |
|---|---|---|
| | on 4.00 mm sieve | on 3.35 mm Sieve |
| SUD | (4.00-4.75 mm) | (3.35-4.00 mm) |
| Number | Hardness (kg) | Hardness (kg) |
| 1 | 30.7 | 19.3 |
| 2 | 34.7 | 29.9 |
| 3 | 32.8 | 26.0 |
| 4 | 30.5 | 27.1 |
| 5 | 30.3 | 22.8 |
| 6 | 28.9 | 25.2 |
| 7 | 33.0 | 22.8 |
| 8 | 29.8 | 24.5 |
| 9 | 26.4 | 24.4 |
| 10 | 28.8 | 26.5 |
| Average | 30.6 | 24.8 |
| % RSD | 7.4 | 10.9 |

### Example 5

Composition and Method of Manufacturing

In Example 5, the composition of the SUDs uses iso propyl alcohol instead of ethanol (as in Example 1). All other steps and procedures remain the same as in Example 1. Table 6 below shows the composition of the various ingredients.

TABLE 6
Formulation composition for batches manufactured
with Isopropyl alcohol: PG

| Ingredient | Weight Units |
|---|---|
| AVICEL PH 101 | 42 |
| Mannitol | 20 |
| Crospovidone | 5 |
| PVP K30 | 3 |
| Propylene Glycol (PG) | 30 |
| Isopropyl Alcohol | 30 |
| Total | 100* |

*Please note that the total is 100 because the 30 weight units contributed by the volatile solvent (isopropyl alcohol) is evaporated off while drying. Therefore, the effective final SUD does not contain any volatile solvent.

### Example 6

Evaluation of SUDs

Dried SUDs from Example 5 were evaluated for the following:

### A. Yield

Classification and yield for SUDs from Example 5 were performed as stated in Example 2. The particle size results for SUDs from Example 5 is shown in Table 7.

TABLE 7
The % yield of the SUDs of various sizes

| Sieve Size Classification | % of SUD |
|---|---|
| (mm) | (bead) weight |
| >5.60 | 0.0 |
| 4.75-5.6 | 0.3 |
| 4.00-4.75 | 2.7 |
| 3.35-4.00 | 15.5 |
| 2.80-3.35 | 28.5 |
| 2.36-2.80 | 18.5 |
| 2.00-2.36 | 11.8 |
| 1.70-2.00 | 7.1 |
| 1.70> | 15.6 |

The combined % yield for the preferred size range (>2.8 mm to <5.6 mm) is 47%.

### B. Hardness of SUDs

Hardness of SUD (bead)s from Example 5 were tested as stated in Example 2. The hardness of SUD (bead)s from the 2.80-3.35 mm and 3.35-4.00 mm size range can be found in Table 8.

TABLE 8
SUDs hardness of preferred sizes of Example
5; RSD: Relative standard deviation

| | SUDs stayed | SUDs that passed |
|---|---|---|
| | on 3.35 mm sieve | through 3.35 mm sieve |
| SUD | (3.35-4.00 mm) | (2.80-3.35 mm) |
| Number | Hardness (kg) | Hardness (kg) |
| 1 | 20.78 | 14.82 |
| 2 | 16.47 | 16.34 |
| 3 | 19.77 | 14.74 |
| 4 | 20.35 | 15.41 |
| 5 | 18.01 | 13.84 |
| 6 | 18.89 | 16.88 |
| 7 | 18.42 | 13.52 |
| 8 | 19.84 | 15.72 |
| 9 | 18.9 | 15.56 |
| 10 | 15.9 | 16.06 |
| Average | 18.73 | 15.29 |
| % RSD | 8.09 | 6.63 |

### Example 7

Composition and Method of Manufacturing

In Example 7, the composition of the SUDs uses water instead of ethanol (as in Example 1). All other steps and procedures remain the same as in in Example 1. Table 9 below shows the composition of the various ingredients.

TABLE 9
Formulation composition for batches manufactured with water: PG

| Ingredient | Weight Units |
|---|---|
| AVICEL PH 101 | 42 |
| Mannitol | 20 |
| Crospovidone | 5 |
| PVP K30 | 3 |
| Propylene Glycol (PG) | 30 |
| Water | 30 |
| Total | 100* |

*Please note that the total is 100 because the 30 weight units contributed by the volatile solvent (water) is evaporated off while drying. Therefore, the effective final SUD does not contain any volatile solvent.

### Example 8

Evaluation of SUDs

Dried SUDs from Example 7 were evaluated for the following:

### A. Yield

Classification and yield for SUDs from Example 7 were performed as stated in Example 2. The particle size results for SUDs from Example 7 is shown in Table 10.

TABLE 10
The % yield of the SUDs of various sizes

| Sieve Size Classification | % of SUD |
|---|---|
| (mm) | (bead) weight |
| >5.60 | 3.3 |
| 4.75-5.6 | 11.1 |
| 4.00-4.75 | 24.5 |
| 3.35-4.00 | 37.0 |
| 2.80-3.35 | 20.5 |
| 2.36-2.80 | 2.7 |
| 2.00-2.36 | 0.2 |
| 1.70-2.00 | 0.0 |
| 1.70> | 0.6 |

The combined % yield for the preferred size range (>2.8 mm to <5.6 mm) is 93.1%.

### B. Hardness of SUDs

Hardness of SUD (bead)s from Example 7 were tested as stated in Example 2. The hardness of SUD (bead)s from the 2.00-2.36 mm, 2.36-2.80 mm, 2.80-3.35 mm and 4.00-4.75 mm size range can be found in Table 11.

TABLE 11
SUDs hardness of preferred sizes of Example
7; RSD: Relative standard deviation

| | SUDs stayed | SUDs that passed |
|---|---|---|
| | on 3.35 mm sieve | through 3.35 mm sieve |
| SUD | (4.00-4.75 mm) | (2.00-3.35 mm) |
| Number | Hardness (kg) | Hardness (kg) |
| 1 | 28.43 | 30.41 |
| 2 | 26.21 | 31.59 |
| 3 | 26.28 | 27.63 |
| 4 | 30.90 | 28.78 |
| 5 | 29.33 | 27.72 |
| 6 | 26.57 | 29.85 |
| 7 | 31.16 | 30.18 |
| 8 | 25.27 | 27.18 |
| 9 | 25.20 | 25.68 |
| 10 | 25.98 | 24.67 |
| Average | 27.53 | 28.37 |
| % RSD | 7.76 | 7.35 |

### Example 9

Composition and Method of Manufacturing

In Example 9, the composition of the SUDs, no volatile liquids were used. All other steps and procedures remain the same as in in Example 1. Table 12 below shows the composition of the SUDs.

TABLE 12
Formulation composition for batches
manufactured with no volatile liquids

| Ingredient | Weight Units |
|---|---|
| AVICEL PH 101 | 42 |
| Mannitol | 20 |
| Crospovidone | 5 |
| PVP K30 | 3 |
| Propylene Glycol (PG) | 60 |
| Water | 0 |

### Example 10

Evaluation of SUDs

SUDs from Example 9 were evaluated for the following:

### A. Yield

Classification and yield for SUDs from Example 9 were performed as stated in Example 2. The particle size results for SUDs from Example 9 is shown in Table 13.

TABLE 13
The % yield of the SUDs of various sizes

| Sieve Size Classification | % of SUD |
|---|---|
| (mm) | (bead) weight |
| >5.60 | 99.8 |
| 4.75-5.6 | 0.2 |
| 4.00-4.75 | 0.0 |
| 3.35-4.00 | 0.0 |
| 2.80-3.35 | 0.0 |
| 2.36-2.80 | 0.0 |
| 2.00-2.36 | 0.0 |
| 1.70-2.00 | 0.0 |
| 1.70> | 0.0 |

The combined % yield for the preferred size range (>2.8 mm to <5.6 mm) is

### B. Hardness of SUDs

Hardness of SUD (bead)s from Example 9 were tested as stated in example 2.

TABLE 14
SUDs hardness of size more than 5.6 mm Example
5; RSD: Relative standard deviation

| SUD | 5.60 mm SUD |
|---|---|
| (bead) Number | (bead)s Hardness (kg) |
| 1 | 21.78 |
| 2 | 22.07 |
| 3 | 22.76 |
| 4 | 25.29 |
| 5 | 22.33 |
| 6 | 22.57 |
| 7 | 22.02 |
| 8 | 27.00 |
| 9 | 20.54 |
| 10 | 20.49 |
| Average | 22.69 |
| % RSD | 8.44 |

### Examples 11-14

Examples 11 to 14 presents the effect of different spheronization speeds on the physical properties of the SUDs.

Composition

554 g batches of SUDs were prepared using Microcrystalline Cellulose (AVICEL PH 101), METHOCEL E5 (METHOCEL™ E5 Premium LV is a Hydroxypropyl Methylcellulose (HPMC) binder), and Water. The formulation composition is shown in Table 15.

TABLE 15
Formulation composition for batches manufactured
with AVICEL, METHOCEL, and Water for Example 11.

| Ingredient | Actual Weight (g) | Weight Units |
|---|---|---|
| AVICEL PH-101 | 525.6 | 96.5 |
| METHOCEL E5 | 28.8 | 3.5 |
| Water | 645.6 | 116.4 |
| Total Nonvolatile Materials | 554.4 | 100 |
| (AVICEL + METHOCEL) | | |

Method of Preparation

In brief, METHOCEL E5 was solubilized in water using HSM-100 Ross High Shear Mixer (Charles Ross and Son Company, 710 Old Willets Path, Hauppauge, N.Y. 11788 U.S.A.) at 1200 RPM to form a binding solution. After 60 seconds of dry mixing, the binding solution was added over AVICEL PH 101 powder in a 6 Quart Professional 600 Series Planetary Mixer (Kitchen Aid, 553 Benson Rd, Benton Harbor, Mich. 49022 U.S.A.), binding solution was poured into the mixer at a constant rate over 90 seconds, while mixing at speed 2. Mixing was continued at speed 2 for 30 seconds. The wet mass was immediately extruded through a 4 mm die using an MG-55 Multi Granulator Extruder (Fuji Paudal, 7-1-45 Nakaishikiri-Cho Higashi Osaka, 579-8014 Japan) at 75 RPM. The output of the extrusion step was spheronized using a QJ-230T Spheronizer (Fuji Paudal, 7-1-45 Nakaishikiri-Cho Higashi Osaka, 579-8014 Japan), fitted with a 5 mm friction plate at various speeds for 2 minutes. The speeds used are given in Table 16.

Resultant SUD (bead)s were dried at room temperature for 48 hours.

TABLE 16
Formulation composition for batches manufactured with
AVICEL, METHOCEL, and Water for Example 11-Example 14.

| Example | Example 11 | Example 12 | Example 13 | Example 14 |
|---|---|---|---|---|
| Spheronization | 1400 rpm | 1200 rpm | 1000 rpm | 800 rpm |
| Speed | | | | |

### Examples 15-18

Evaluation of SUDs

Dried SUDs from examples 11-14 were evaluated for yield.
Classification and yield for SUDs from examples 11-14 were performed as stated in example 2. Results are given in Table 17.

TABLE 17
Classification yield analysis for example 11-14

| | Example 11 | | Example 12 | | Example 13 | | Example 14 | |
|---|---|---|---|---|---|---|---|---|
| | 1400 RPM | | 1200 RPM | | 1000 RPM | | 800 RPM | |
| | % of | | % of | | % of | | % of | |
| Sieve Size | SUD | | SUD | | SUD | | SUD | |
| Classification | (bead) | Avg wt | (bead) | Avg wt | (bead) | Avg wt | (bead) | Avg wt |
| (mm) | wt | (mg) | wt | (mg) | wt | (mg) | wt | (mg) |
| >5.60 | 0 | 0 | 0.04 | 133 | 0 | 0 | 0 | 0 |
| 4.75-5.6 | 0 | 0 | 0.50 | 84 | 0.04 | 66 | 0 | 0 |
| 4.00-4.75 | 4 | 50 | 4.5 | 53 | 5 | 50 | 13 | 50 |
| 3.35-4.00 | 31 | 31 | 28 | 30 | 36 | 32 | 50 | 32 |
| 2.80-3.35 | 33 | 19 | 35 | 21 | 33 | 20 | 23 | 19 |
| 2.36-2.80 | 6 | 11 | 16 | 13 | 13 | 14 | 6 | 12 |
| 2.00-2.36 | 7 | 8 | 8 | 8 | 5 | 8 | 2 | 8 |
| 1.70-2.00 | 5 | 5 | 4 | 5 | 1 | 5 | 1 | 4 |
| 1.70> | 15 | 1 | 4 | 2 | 6 | 2 | 4 | 2 |

### Examples 19 and 20

Examples 19 and 20 present the effect of different spheronization times on the physical properties of the SUDs.

Composition

544 g batches of SUDs were prepared using Microcrystalline Cellulose (AVICEL PH 101) (43.8% w/w), METHOCEL E5 (1.6% w/w), and Water (54.6% w/w). The formulation composition is shown in Table 18.

TABLE 18
Formulation composition for batches manufactured
with AVICEL, METHOCEL, and water for Example 11.

| Ingredient | Weight (g) | Weight Units |
|---|---|---|
| AVICEL PH-101 | 525.6 | 96.5 |
| METHOCEL E5 | 28.8 | 3.5 |
| Water | 645.6 | 116.4 |
| Total Nonvolatile Materials | 554.4 | 100 |
| (AVICEL + METHOCEL) | | |

Method of Preparation

METHOCEL E5 was solubilized in water using HSM-100 Ross High Shear Mixer (Charles Ross and Son Company, 710 Old Willets Path, Hauppauge, N.Y. 11788 U.S.A.) at 1200 RPM to form a binding solution. After 60 seconds of dry mixing, the binding solution was added over AVICEL PH 101 powder in a 6 Quart Professional 600 Series Planetary Mixer (Kitchen Aid, 553 Benson Rd, Benton Harbor, Mich. 49022 U.S.A.), binding solution was poured into the mixer at a constant rate over 90 seconds, while mixing at speed 2. Mixing was continued at speed 2 for 30 seconds. The wet mass was immediately extruded through a 4 mm die using an MG-55 Multi Granulator Extruder (Fuji Paudal, 7-1-45 Nakaishikiri-Cho Higashi Osaka, 579-8014 Japan) at 75 RPM. The output of the extrusion step was spheronized using a QJ-230T Spheronizer (Fuji Paudal, 7-1-45 Nakaishikiri-Cho Higashi Osaka, 579-8014 Japan), fitted with a 5 mm friction plate at 1400 (example 19) and 1000 rpm (example 20) speeds with samples taken every 30 seconds for 4 minutes. Resultant SUD (bead)s were dried at room temperature for 48 hours.

### Examples 21 and 22

Dried SUDs from Examples 19 and 20 were Evaluated for Yield.

Classification and yield for SUDs from examples 19 and 20 were performed as stated in example 2. Results are given in Table 19 and Table 20.

TABLE 19
Sieve and weight analysis for batches manufactured with AVICEL,
METHOCEL, and Water ranging from a spheronization speeds
of 1400 RPM collected every 30 seconds (example 19)
a.:

| | 1400 RPM for | | 1400 RPM for | | 1400 RPM for | | 1400 RPM for | |
|---|---|---|---|---|---|---|---|---|
| Sieve Size | 30 seconds | | 1 minute | | 1.5 minutes | | 2 minutes | |
| Classification | | Avg wt | | Avg wt | | Avg wt | | Avg wt |
| (mm) | % wt | (mg) | % wt | (mg) | % wt | (mg) | % wt | (mg) |
| >5.60 | 0 | 0 | 0 | 0 | 0 | 0 | 0 | 0 |
| 4.75-5.6 | 0 | 0 | 0.4 | 69 | 0.2 | 74 | 0 | 0 |
| 4.00-4.75 | 8 | 45 | 13 | 41 | 10 | 45 | 1.4 | 90 |
| 3.35-4.00 | 27 | 30 | 25 | 29 | 26 | 33 | 13 | 32 |
| 2.80-3.35 | 25 | 18 | 22 | 18 | 26 | 20 | 26 | 19 |
| 2.36-2.80 | 11 | 11 | 11 | 12 | 10 | 12 | 21 | 13 |
| 2.00-2.36 | 6 | 7 | 7 | 7 | 7 | 7 | 12 | 8 |
| 1.70-2.00 | 3 | 5 | 4 | 4 | 4 | 4 | 6 | 5 |
| 1.70> | 19 | 2 | 19 | 2 | 16 | 2 | 20 | 0.4 |

b.:

| | 1400 RPM for | | 1400 RPM for | | 1400 RPM for | | 1400 RPM for | |
|---|---|---|---|---|---|---|---|---|
| Sieve Size | 2.5 minutes | | 3 minutes | | 3.5 minutes | | 4 minutes | |
| Classification | | Avg wt | | Avg wt | | Avg wt | | Avg wt |
| (mm) | % wt | (mg) | % wt | (mg) | % wt | (mg) | % wt | (mg) |
| >5.60 | 0 | 0 | 0 | 0 | 0 | 0 | 0 | 0 |
| 4.75-5.6 | 2 | 73 | 0 | 0 | 0 | 0 | 0 | 0 |
| 4.00-4.75 | 14 | 34 | 1 | 52 | 0 | 0 | 1 | 49 |
| 3.35-4.00 | 30 | 31 | 7 | 32 | 10 | 32 | 15 | 34 |
| 2.80-3.35 | 19 | 21 | 32 | 21 | 30 | 22 | 36 | 21 |
| 2.36-2.80 | 11 | 12 | 22 | 13 | 23 | 13 | 21 | 13 |
| 2.00-2.36 | 5 | 8 | 12 | 8 | 14 | 8 | 11 | 8 |
| 1.70-2.00 | 0.3 | 5 | 7 | 5 | 7 | 5 | 5 | 5 |
| 1.70> | 19 | 1 | 19 | 1 | 17 | 1 | 12 | 2 |

TABLE 20
Sieve and weight analysis for batches manufactured with AVICEL,
METHOCEL, and Water ranging from a spheronization speeds
of 1000 RPM collected every 30 seconds (example 20)
a.:

| | 1000 RPM for | | 1000 RPM for | | 1000 RPM for | | 1000 RPM for | |
|---|---|---|---|---|---|---|---|---|
| Sieve Size | 30 seconds | | 1 minute | | 1.5 minutes | | 2 minutes | |
| Classification | | Avg wt | | Avg wt | | Avg wt | | Avg wt |
| (mm) | % wt | (mg) | % wt | (mg) | % wt | (mg) | % wt | (mg) |
| >5.60 | 0 | 0 | 0 | 0 | 0 | 0 | 0 | 0 |
| 4.75-5.6 | 0 | 0 | 0 | 0 | 0 | 0 | 0 | 0 |
| 4.00-4.75 | 0 | 0 | 0 | 0 | 0.2 | 47 | 0 | 0 |
| 3.35-4.00 | 4 | 30 | 10 | 31 | 13 | 33 | 14 | 32 |
| 2.80-3.35 | 50 | 22 | 49 | 23 | 45 | 23 | 43 | 23 |
| 2.36-2.80 | 21 | 13 | 19 | 14 | 21 | 14 | 21 | 13 |
| 2.00-2.36 | 8 | 8 | 7 | 9 | 8 | 8 | 9 | 9 |
| 1.70-2.00 | 2 | 5 | 2 | 6 | 2 | 5 | 3 | 6 |
| 1.70> | 16 | 0.3 | 12 | 1 | 11 | 0.5 | 10 | 1 |

b:

| | 1000 RPM for | | 1000 RPM for | | 1000 RPM for | | 1000 RPM for | |
|---|---|---|---|---|---|---|---|---|
| Sieve Size | 2.5 minutes | | 3 minutes | | 3.5 minutes | | 4 minutes | |
| Classification | | Avg wt | | Avg wt | | Avg wt | | Avg wt |
| (mm) | % wt | (mg) | % wt | (mg) | % wt | (mg) | % wt | (mg) |
| >5.60 | 0 | 0 | 0 | 0 | 0 | 0 | 0 | 0 |
| 4.75-5.6 | 0 | 0 | 0 | 0 | 0 | 0 | 0 | 0 |
| 4.00-4.75 | 0 | 0 | 0 | 0 | 0 | 0 | 0.3 | 45 |
| 3.35-4.00 | 16 | 34 | 17 | 32 | 18 | 31 | 24 | 32 |
| 2.80-3.35 | 45 | 21 | 46 | 22 | 44 | 21 | 49 | 25 |
| 2.36-2.80 | 18 | 13 | 19 | 13 | 21 | 13 | 16 | 13 |
| 2.00-2.36 | 9 | 9 | 9 | 8 | 8 | 9 | 6 | 8 |
| 1.70-2.00 | 2 | 6 | 2 | 5 | 3 | 6 | 2 | 8 |
| 1.70> | 9 | 1 | 8 | 1 | 7 | 1 | 3 | 1 |

### Example 23

Composition

A 300 g batch of Acetaminophen containing SUD was prepared using the formula: Microcrystalline Cellulose (AVICEL PH 101) (42% w/w), Acetaminophen (APAP) (50% w/w) Croscarmellose Sodium (5% w/w) (internally cross-linked sodium carboxymethylcellulose for use as a superdisintegrant in pharmaceutical formulations), and Hydroxy propyl methylcellulose (HPMC) E5 (6% w/w). This example presents the SUD (bead) size prepared with a loading disintegrant. The composition of example 23 is shown in Table 21.

TABLE 21
Formulation composition for batches manufactured
with AVICEL, Acetaminophen, Croscarmellose
Sodium, HPMC E5, and water (Example 23).

| Ingredient | Weight Units | Actual Weights (g) |
|---|---|---|
| AVICEL PH 101 | 39 | 117.2 |
| Acetaminophen | 50 | 150.1 |
| Croscarmellose Sodium | 5 | 15.0 |
| HPMC E5 | 6 | 18.0 |
| Water* | 41.1* | 246.5* |
| Total Nonvolatile Materials | 100 | 300 |

Method of Preparation

In brief, AVICEL PH 101, APAP, Croscarmellose Sodium and HPMC E5 were mixed in a 6 Quart Professional 600 Series Planetary Mixer (Kitchen Aid, 553 Benson Rd, Benton Harbor, Mich. 49022 U.S.A.) for 3 minutes at a speed of 2, water was poured into the mixer at a constant rate over 90 seconds, while mixing at speed 2. Mixing was continued at speed 2 for 1 minute. The wet mass was immediately placed in the oven at 50° C. for two hours and dried to 41.1% water. The mass was then extruded through a 4 mm die using an MG-55 Multi Granulator Extruder (Fuji Paudal, 7-1-45 Nakaishikiri-Cho Higashi Osaka, 579-8014 Japan). The output of the extrusion step was spheronized using a QJ-230T Spheronizer (Fuji Paudal, 7-1-45 Nakaishikiri-Cho Higashi Osaka, 579-8014 Japan), fitted with a 5 mm friction plate for 4 minutes at 600 rpm. Resultant SUD (bead)s were dried in the oven at 50° C. for 24 hours.

### Example 24

Evaluation of SUDs

Dried SUDs from Example 23 were evaluated for classification and yield as stated in example 2. the average SUD weight for each size range is shown in Table 22.

TABLE 22
SUP (bead) size distribution for the APAP for batches
manufactured with AVICEL, Acetaminophen, Croscarmellose
Sodium, HPMC E5, and water (Example 24).

| | |
|---|---|
| Sieve Size | Example 24 |

| Classification | SUD (bead) Size | Average SUD (bead) |
|---|---|---|
| (mm) | Distribution (%) | Weight (mg) |
| >5.60 | 0.0 | N/A |
| 4.75-5.6 | 13.5 | 65 |
| 4.00-4.75 | 49.8 | 46 |
| 3.35-4.00 | 25.2 | 29 |
| 2.80-3.35 | 7.9 | 20 |
| 2.36-2.80 | 1.8 | 11 |
| 2.00-2.36 | 0.9 | 6 |
| 1.70-2.00 | 0.5 | 4 |
| 1.70> | 0.3 | N/A |

SUD (bead)s from each size were assayed and dosage was determined. The dosage of the active ingredient was calculated by grinding 10 SUDs and assaying the extract using HPLC. The results are given in Table 23.

TABLE 23
Acetaminophen dosage for each SUD (bead) size from Example 25

| | Example 25 |
|---|---|
| Sieve Size Classification | Dosage (μg of Acetaminophen |
| (mm) | per SUD (bead)) |
| 4.75-5.6 | 32 |
| 4.00-4.75 | 20 |
| 3.35-4.00 | 13 |
| 2.80-3.35 | 10 |
| 2.36-2.80 | 5 |
| 2.00-2.36 | 3 |
| 1.70-2.00 | 2 |

### Example 25

Composition

A 744 g batch of SUDs with levothyroxine sodium hydrate and a self-emulsifying drug delivery system was prepared using the formula in Table 24.

TABLE 24
Formulation composition for batches manufactured
with levothyroxine sodium hydrate in a self-
emulsifying drug delivery systems (SEDDs).

| | |
|---|---|
| Ingredient | Weight Units |

| | | |
|---|---|---|
| SEDDs | Propylene Glycol | 10 |
| Concentrate | Poloxamer 124 | 5 |
| | Butylated Hydroxyanisole (BHA) | 0.33 |
| | Butylated Hydroxytoluene (BHT) | 0.03 |
| | Levothyroxine Sodium Hydrate | 0.11 |
| | Hydrophilic fumed silica like | 7.5 |
| | AEROSIL 200 | |
| Dry Blend | AVICEL PH 101 | 28.9 |
| | Mannitol | 20 |
| | Croscarmellose Sodium | 5 |
| | Crospovidone | 5 |
| Binder | Butylated Hydroxytoluene | 0.1 |
| Solution | Propylene Glycol | 15 |
| | Ethanol | 24 |
| | PVP | 3 |

| | |
|---|---|
| Total Non-Voatile Materials | 100 |

*Please note that the total is 100 because the 24 weight units contributed by the volatile solvent (ethanol) is evaporated off while drying. Therefore, the effective final SUD does not contain any volatile solvent.

Method of Preparation

In brief, propylene glycol, poloxamer 124, BHA and BHT are mixed in a beaker using a top stirrer (IKA Eurostar 20, 79219 Staufen, Germany) at 500 rpm for 15 minutes. The solution was purged with nitrogen for another 15 minutes. Levothyroxine sodium hydrate was added to the solution and mixed with the top stirrer for another 20 minutes at 500 rpm. The solution was then poured over hydrophilic fumed silica like AEROSIL 200 and mixed in a mortar and pestle. This forms the SEDDs concentrate. BHT, and PVP K30 were dissolved in ethanol (200 proof) using an HSM-100 Ross High Shear Mixer (Charles Ross and Son Company, 710 Old Willets Path, Hauppauge, N.Y. 11788 U.S.A.). Propylene glycol was added to the solution and mixed with the HSM-100 Ross High Shear Mixer. This formed the binder solution. AVICEL PH 101, mannitol, croscarmellose sodium and crospovidone were mixed in a 6 Quart Professional 600 Series Planetary Mixer (Kitchen Aid, 553 Benson Rd, Benton Harbor, Mich. 49022 U.S.A) for 3 minutes at a speed of 2, binder solution was poured into the mixer at a constant rate over 90 seconds, while mixing at speed 2. Mixing was continued at speed 2 for 1 minute. The SEDDs concentrate was added to the wet mass and mixed for another minute. The mass was then extruded through a 4 mm die using an MG-55 Multi Granulator Extruder (Fuji Paudal, 7-1-45 Nakaishikiri-Cho Higashi Osaka, 579-8014 Japan). The output of the extrusion step was spheronized using a QJ-230T Spheronizer (Fuji Paudal, 7-1-45 Nakaishikiri-Cho Higashi Osaka, 579-8014 Japan), fitted with a 5 mm friction plate for 4 minutes at 600 rpm. Resultant SUD (bead)s were dried at room temperature for 17 hours.

### Example 26

Evaluation of SUDs

Dried SUDs from Example 25 were evaluated for the following:

### A. Yield

Classification and yield for SUDs from example 25 were performed as stated in example 2. The particle size results for SUDs from example 25 is shown in Table 25.

TABLE 25
SUD (bead) size distribution for SUDs manufacture with
levothyroxine sodium hydrate and SEDDs (Example 25).

| | |
|---|---|
| Sieve Size | Example 25 |

| Classification | SUD (bead) Size | Average SUD |
|---|---|---|
| (mm) | Distribution (%) | (bead) Weight (mg) |
| >5.60 | 2.2 | 157 |
| 4.75-5.6 | 20.8 | 82 |
| 4.00-4.75 | 42.9 | 57 |
| 3.35-4.00 | 21.6 | 37 |
| 2.80-3.35 | 7.7 | 21 |
| 2.36-2.80 | 2.7 | 12 |
| 2.00-2.36 | 1.4 | 8 |
| 1.70-2.00 | 0.5 | 5 |
| 1.70> | 0.2 | N/A |

SUD (bead)s from each tray were assayed and dosage was determined using HPLC. The results are given in Table 26.

TABLE 26
Levothyroxine sodium (anhydrous) dosage
for each SUD (bead) size from Example 25.

| | Example 25 |
|---|---|
| Sieve Size Classification | Dosage (mg of Levothyroxine |
| (mm) | Sodium per SUD (bead)) |
| >5.60 | 137 |
| 4.75-5.6 | 71 |
| 4.00-4.75 | 49 |
| 3.35-4.00 | 31 |
| 2.80-3.35 | 18 |
| 2.36-2.80 | 10 |
| 2.00-2.36 | 7 |
| 1.70-2.00 | 4 |

### B. Hardness of SUDS

Hardness of SUD (bead)s from Example 23 were tested as stated in Example 2. The hardness of SUD (bead)s from the 3.35-4.00 mm and 4.00-4.75 mm size range can be found in Table 27.

TABLE 27
SUDs hardness of preferred size ranges of Example 3.

| | SUDs stayed on | SUDs stayed on | SUDs stayed on | SUDs stayed on |
|---|---|---|---|---|
| SUD | 4.75 mm Sieve | 4.00 mm Sieve | 3.35 mm Sieve | 2.80 mm Sieve |
| Number | Hardness (kg) | Hardness (kg) | Hardness (kg) | Hardness (kg) |
| 1 | 31.059 | 26.414 | 19.995 | 19.546 |
| 2 | 32.754 | 23.867 | 20.320 | 19.916 |
| 3 | 29.833 | 26.555 | 19.677 | 19.612 |
| 4 | 31.365 | 28.220 | 21.990 | 19.406 |
| 5 | 27.620 | 25.367 | 20.593 | 19.703 |
| 6 | 29.588 | 23.769 | 20.769 | 18.931 |
| 7 | 30.893 | 24.698 | 23.206 | 18.002 |
| 8 | 28.430 | 27.812 | 18.505 | 18.808 |
| 9 | 30.982 | 25.117 | 20.023 | 19.333 |
| 10 | 30.293 | 22.427 | 21.844 | 19.361 |
| Average | 30.282 | 25.425 | 20.692 | 19.262 |
| % RSD | 1.489 | 1.842 | 1.343 | 0.554 |

### Example 27

Preparation of Package with Multiple Chambers Containing Drug

The various drug dosages of active ingredients obtained in a single process as shown in the previous examples are then directly dispensed in various chambers in the same package. Hence the package obtained has a plurality of chambers each of the plurality of chambers comprises a solid unit dosage comprising an active ingredient. The package is configured to provide an individualized delivery of the active ingredient in a plurality of different concentrations.

1. A package comprising a plurality of chambers, each of the plurality of chambers comprising a solid unit dosage comprising an active ingredient, wherein the package is configured to provide an individualized delivery of the active ingredient in a plurality of different concentrations of the active ingredient to a human or an animal, wherein a concentration of the active ingredient in the solid unit dosage in each of the plurality of chambers is different, wherein the plurality of different concentrations of the active ingredient comprise at least two different concentrations that are greater than zero weight percent of the active ingredient.

2. The package of claim 1, wherein an effective amount of the active ingredient is present in the solid unit dosage according to a size or a weight of the solid unit dosage.

3. The package of claim 1, wherein the solid unit dosage is neither a capsule nor a tablet.

4. The package of claim 1, wherein the solid unit dosage further comprises a safe and effective amount of at least one substance selected from the group consisting of a filler, a carrier, a binder, a disintegrant, a surface active agent, a lubricant and combinations thereof.

5. The package of claim 1, wherein the solid unit dosage further comprises a coating material.

6. The package of claim 1, wherein the active ingredient comprises a material comprising a therapeutic agent, a diagnostic agent, a food supplement or combinations thereof, and the material is present in an amount of 0.01 to 80 percent by weight of the solid unit dosage.

7. The package of claim 1, wherein the solid unit dosage comprises a suitable medium comprising a volatile liquid or a non-volatile liquid that is present in an amount of 1 to 75 percent by weight of the solid unit dosage.

8. The package of claim 7, wherein the volatile liquid is selected from the group consisting of water, methanol, ethanol, 1-propanol, isopropyl alcohol and combinations thereof.

9. The package of claim 7, wherein the non-volatile liquid comprises polyethylene glycol.

10. The package of claim 4, wherein the filler is selected from the group consisting of mannitol, lactose, a polymeric saccharide, starch, polyvinylpyrrolidone and combinations thereof, wherein the filler is present in an amount of 0.01 to 40 percent by weight of the solid unit dosage.

11. The package of claim 4, wherein the carrier is selected from the group consisting of silicon dioxide, zinc oxide, magnesium oxide, a porous natural polysaccharide, a synthetic polysaccharide and combinations thereof, wherein the carrier is present in an amount of 0.01 to 50 percent by weight of the solid unit dosage.

12. The package of claim 4, wherein the binder is selected from the group consisting of hydroxypropyl methylcellulose, polyvinyl pyrrolidone, methyl cellulose, gelatin, starch, sucrose, lactose and combinations thereof, wherein the binder is present in an amount of 0.01 to 30 percent by weight of the solid unit dosage.

13. The package of claim 4, wherein the disintegrant is selected from the group consisting of cross-caramellose, cross-povidone, a modified starch, sodium starch glycolate and combinations thereof, wherein the disintegrant is present in an amount of 0.01 to 15 percent by weight of the solid unit dosage.

14. The package of claim 4, wherein the surface active agent comprises polaxmer, wherein the surface active agent is present in an amount of 0.01 to 50 percent by weight of the solid unit dosage.

15. The package of claim 1, wherein the solid unit dosage has following properties:
(a) a sphericity factor of about 0.9 to about 1.0;
(b) a hardness more than 10 kilograms;
(c) a diameter in a range from 1.5 to 6 mm;
(d) a weight in a range from 15 to 300 mg.

16. The package of claim 1, wherein the plurality of chambers comprises at least three chambers, and wherein at least one of the plurality of chambers comprises a placebo having zero weight percent of the active ingredient.

17. A method comprising mixing an active ingredient in a suitable medium, and forming a solid unit dosage by extrusion and spheronization, wherein the solid unit dosage comprises the active ingredient dispersed in the suitable medium, wherein an effective amount of the active ingredient is present in the solid unit dosage according to a size or a weight of the solid unit dosage such that the solid unit dosage is configured to provide an individualized delivery of the active ingredient to a human or an animal.

18. The method of claim 17, wherein the method further comprises drying using vacuum, heat, air, or any pharmaceutically acceptable inert gas and coating the solid unit dosage.

19. The method of claim 17, wherein the method comprises a continuous process or a batch process that simultaneously produces at least two strengths of the active ingredient.

20. A delivery system comprising a solid unit dosage comprising an active ingredient, wherein the delivery system is configured to provide an individualized delivery of the active ingredient in a plurality of different concentrations of the active ingredient to a human or an animal, wherein a concentration of the active ingredient in the solid unit dosage within the delivery system is different, wherein the plurality of different concentrations of the active ingredient comprise at least two different concentrations that are greater than zero weight percent of the active ingredient.
